(19) United States
(12) Patent Application Publication
(10) Pub. No.: US 2013/0302414 A1
(43) Pub. Date: Nov. 14, 2013

(54) SOLUBILIZED CAPSULE FORMULATION OF 1,1-DIMETHYLETHYL [(1S)-1-CARBAMOYL)PYRROLIDIN-1-YL]CARBONYL}-2,2-DIMETHYLPROPYL]CARBAMATE

(71) Applicant: Bristol-Myers Squibb Company, (US)

(72) Inventor: Robert Kevin Perrone, Belle Mead, NJ (US)

(21) Appl. No.: 13/872,652

(22) Filed: Apr. 29, 2013

Related U.S. Application Data
(60) Provisional application No. 61/643,454, filed on May 7, 2012.

(51) Int. Cl. A61K 9/48; A61K 38/06

(52) U.S. Cl. CPC A61K 9/48; A61K 38/06
USPC 424/451; 514/21.9; 514/4.3

(57) ABSTRACT

The present disclosure includes various embodiments directed to a solubilized capsule formulation of asunaprevir, 1,1-dimethylethyl[(1S)-1-{[(2S,4R)-4-(7-chloro-4methoxyisoquinolin-1-yloxy)-2-({(1R,2S)-1-[(cyclopropylsulfonyl)carbamoyl]-2-ethenylcyclopropyl}carbamoyl)pyrrolidin-1-yl]carbonyl}-2,2-dimethylpropyl]carbamate, and to methods including asunaprevir.

### CROSS-REFERENCE TO RELATED APPLICATIONS

This application claims the benefit of U.S. Provisional Application Ser. No. 61/643,454 filed May 7, 2012.

The present disclosure relates generally to a solubilized capsule dosage formulation for poorly water soluble pharmaceutical compounds that have shown low oral bioavailability with a significant food effect. In particular, the disclosure relates to a new solubilized capsule dosage formulation of asunaprevir, 1,1-dimethylethyl [(1S)-1-{[(2S,4R)-4-(7-chloro-4methoxyisoquinolin-1-yloxy)-2-({(1R,2S)-1-[(cyclopropylsulfonyl)carbamoyl]-2-ethenylcyclopropyl}carbamoyl)pyrrolidin-1-yl]carbonyl}-2,2-dimethylpropyl]carbamate, and to methods of using the formulation for the treatment and/or inhibition of the hepatitis C virus and infections caused thereby.

The compound of formula (I), asunaprevir, 1,1-dimethylethyl [(1S)-1-{[(2S,4R)-4-(7-chloro-4methoxyisoquinolin-1-yloxy)-2-({(1R,2S)-1-[(cyclopropylsulfonyl)carbamoyl]-2-ethenylcyclopropyl}carbamoyl)pyrrolidin-1-yl]carbonyl}-2,2-dimethylpropyl]carbamate, is a selective NS3 protease inhibitor and is useful in the treatment of the hepatitis C virus (HCV).

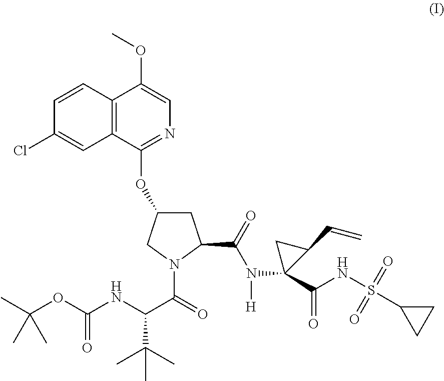 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

Asunaprevir and its preparation have been previously described in U.S. Pat. No. 6,995,174, issued Feb. 7, 2006, U.S. Pat. No. 7,449,479, issued Nov. 11, 1988, and U.S. Pat. No. 7,915,291 which issued Mar. 29, 2011.

Asunaprevir is a selective NS3 protease inhibitor of hepatitis C virus (HCV), and is intended for the oral treatment of hepatitis C infection. Numerous solid dosage forms of asunaprevir such as dry and wet granulations of amorphous or crystalline forms of the drug in a capsule or compressed into a tablet have shown low oral bioavailability with significant food effect. In addition, a significant reduction in the crystallinity of asunaprevir and formation of amorphous drug substance has been observed that results from various unit operations (e.g., roller compaction, tablet compression) in the manufacturing processes for capsules and tablets manufactured from dry and wet granulations and powders. Furthermore, the drug substance is susceptible to conversion from anhydrous to hydrate (or vice-versa) crystalline forms depending on the relative humidity of the environment during manufacture of the capsules or tablets from dry and wet granulations or powders. These factors significantly complicate the manufacturing process and control of the drug product due to potential variability in the drug form present in the formulation, requiring an increased level of monitoring, testing and controls.

Suitably stable dosage forms of asunaprevir with significantly enhanced oral bioavailability and elimination of the food effect were obtained by solubilizing the drug in a mixture of excipients which include lipid components and filling into capsules. These dosage forms readily provide the drug in an emulsified-state at high concentrations which resist drug precipitation following dilution in aqueous milieu, significantly enhancing the oral absorption of asunaprevir in the fasted-state and providing comparable absorption in the fed-state.

The process for manufacturing the solubilized formulations of asunaprevir consists of significantly less unit operations compared to manufacturing processes for capsules or tablets from dry and wet granulations or powders. In addition, the solubilized-state of the drug eliminates the need for monitoring, testing and control of the drug substance for potential changes or variability in the form induced by the processes used for manufacturing capsules or tablets from dry and wet granulations or powders.

In its first aspect the present disclosure provides a formulation comprising a capsule comprising at least one active pharmaceutical ingredient comprising a solubilized compound having the formula (I)

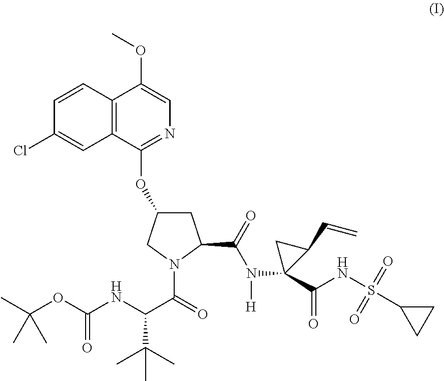 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

In a first embodiment of the first aspect the formulation further comprises at least one solubilizer and optionally comprises at least one surfactant, and/or at least one stabilizer. In a second embodiment of the first aspect the at least one stabilizer is included in the range from about 0.01 to about 1% w/w.

In a third embodiment of the first aspect the at least one active pharmaceutical ingredient is included in the range from about 0.1 to about 60% w/w, the at least one solubilizer is included in the range from about 1 to about 80% w/w, the at least one surfactant is included in the range from about 0 to about 60% w/w, and the at least one stabilizer is included in the range from about 0.01 to about 1% w/w.

In a fourth embodiment of the first aspect the at least one active pharmaceutical ingredient is included in the range from about 1 to about 40% w/w, the at least one solubilizer is included in the range from about 10 to about 80% w/w, the at least one surfactant is included in the range from about 5 to about 40% w/w, and the at least one stabilizer is included in the range from about 0.02 to about 0.5% w/w.

In a fifth embodiment of the first aspect the at least one active pharmaceutical ingredient is included in the range from about 1 to about 40% w/w, the at least one solubilizer is included in the range from about 5 to about 80% w/w, the at least one surfactant is included in the range from about 15 to about 40% w/w, and the at least one stabilizer is included in the range from about 0.05 to about 0.2% w/w.

In a sixth embodiment of the first aspect the at least one solubilizer is comprised of medium-chain fatty acid triglycerides and a combination of medium-chain fatty acid mono- and diglycerides.

In a seventh embodiment of the first aspect the at least one solubilizer is polyoxyethylated glycerides.

In an eighth embodiment of the first aspect the at least one surfactant is polyoxyethylene sorbitan monooleate.

In a ninth embodiment of the first aspect the at least one stabilizer is butylated hydroxytoluene.

In a second aspect the present disclosure provides a formulation comprising:

(a) at least one active pharmaceutical ingredient wherein the at least one active pharmaceutical ingredient comprises a compound of formula (I)

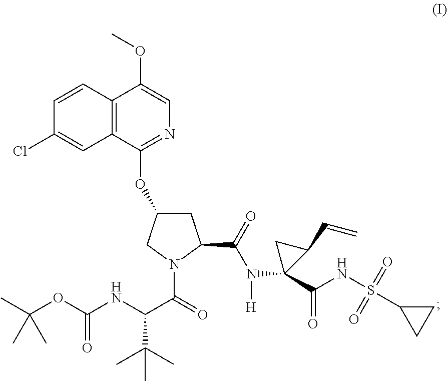 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

(b) at least one solubilizer selected from medium-chain fatty acid triglycerides, polyoxyethylated glycerides, a combination of medium-chain fatty acid mono- and diglycerides, and combinations thereof;

(c) at least one surfactant which is polyoxyethylene sorbitan monooleate; and

(d) at least one stabilizer which is butylated hydroxytoluene.

In a first embodiment of the second aspect the at least one active pharmaceutical ingredient is included in the range from about 1 to about 40% w/w.

In a second embodiment of the second aspect the at least one stabilizer is included in the range from about 0.05 to about 0.2% w/w.

In a third embodiment of the second aspect the at least one solubilizer is medium-chain fatty acid triglycerides and a combination of medium-chain fatty acid mono- and diglycerides. In a fourth embodiment the at least one solubilizer is included in the range from about 10 to about 80% w/w. In a fifth embodiment the at least one surfactant is included in the range from about 15 to about 40% w/w.

In a fifth embodiment of the second aspect the at least one solubilizer is polyoxyethylated glycerides. In a sixth embodiment the at least one solubilizer is included in the range from about 1 to about 80% w/w. In a seventh embodiment the at least one surfactant is included in the range from about 1 to about 40% w/w.

In a third aspect the present disclosure provides a formulation comprising a capsule comprising at least one active pharmaceutical ingredient comprising a solubilized compound having the formula (I)

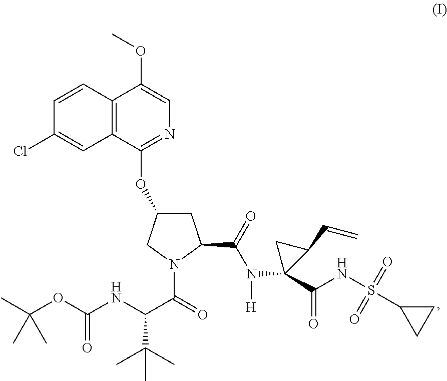 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

that is stable for at least six months at 40° C. and 75% relative humidity.

In a first embodiment of the third aspect the at least one active pharmaceutical ingredient has at least about 98% potency retention.

In a second embodiment of the third aspect the formulation has less than 10% total degradants.

In a third embodiment of the third aspect the formulation has less than 5% total degradants.

In a fourth embodiment of the third aspect the formulation has less than 2% total degradants.

In a fifth embodiment of the third aspect the formulation has less than 1% total degradants.

In a sixth embodiment of the third aspect the formulation has less than 0.5% total degradants.

In a seventh embodiment of the third aspect the formulation comprises at least one stabilizer in an amount from about 0.01 to about 1.0% w/w. In an eighth embodiment the at least one stabilizer is selected from butylated hydroxytoluene, butylated hydroxyanisole, Vitamin E, propyl gallate, ascorbic acid, and tert-butylhydroquinone. In a ninth embodiment the at least one stabilizer comprises butylated hydroxytoluene.

In a fourth aspect the present disclosure provides a method of administering a formulation comprising orally administering to a fasted or fed mammalian subject a formulation comprising Compound (I) having the formula:

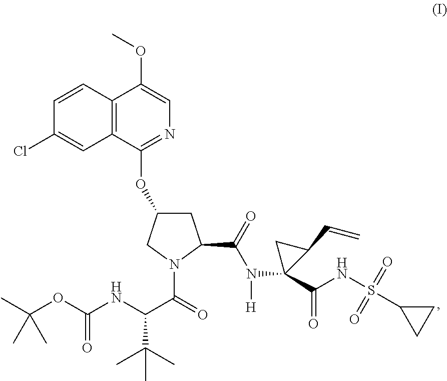 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

to provide a total blood plasma concentration profile of Compound (I), as measured by AUC at 24 hours after an initial dose of the composition, that is at least greater than about 50% of the total blood plasma concentration as measured by AUC at 24 hours of an initial dose of an orally administered solution comprising Compound (I).

In a first embodiment of the fourth aspect the AUC is at least greater than about 60% of the AUC at 24 hours of the solution when orally administered regardless if the subject is fasted or fed.

In a second embodiment of the fourth aspect the AUC is at least greater than about 70% of the AUC at 24 hours of the solution when orally administered regardless if the subject is fasted or fed.

In a third embodiment of the fourth aspect the AUC is at least greater than about 80% of the AUC at 24 hours of the solution when orally administered regardless if the subject is fasted or fed.

In a fourth embodiment of the fourth aspect the AUC is at least greater than about 90% of the AUC at 24 hours of the solution when orally administered regardless if the subject is fasted or fed.

In a fifth embodiment of the fourth aspect the AUC is at least greater than about 93% of the AUC at 24 hours of the solution when orally administered regardless if the subject is fasted or fed.

In a sixth embodiment of the fourth aspect the AUC is at least greater than about 95% of the AUC at 24 hours of the solution when orally administered regardless if the subject is fasted or fed.

In a seventh embodiment of the fourth aspect the formulation is in a form of a capsule.

In an eighth embodiment of the fourth aspect the formulation is in a form of a solubilized capsule.

In a fifth aspect the present disclosure provides a method of administering a formulation comprising orally administering to a fasted mammalian subject the composition comprising Compound (I) having the formula:

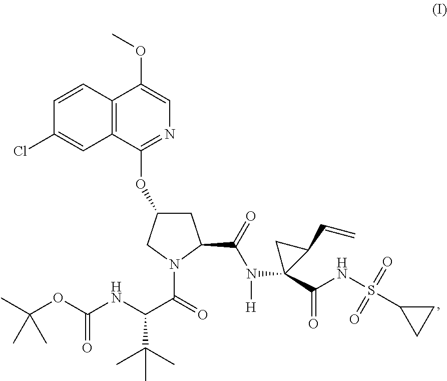 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

to provide a blood plasma concentration profile after an initial dose of the composition with a Cmax of Compound (I) after an initial dose of the composition that is at least greater than about 40% of the Cmax of an orally administered solution comprising Compound (I).

In a first embodiment of the fifth aspect the Cmax of the composition is at least or greater than about 50% of the Cmax of an orally administered solution.

In a second embodiment of the fifth aspect the Cmax of the composition is at least or greater than about 60% of the Cmax of an orally administered solution.

In a third embodiment of the fifth aspect the Tmax is at least about 3 hours.

In a fourth embodiment of the fifth aspect the formulation is in a form of a capsule.

In a fifth embodiment of the fifth aspect the formulation is in a form of a solubilized capsule.

In a sixth aspect the present disclosure provides a method of administering an oral solid dosage composition comprising orally administering to a fasted mammalian subject the composition comprising at least one poorly soluble active pharmaceutical ingredient to provide a total blood plasma concentration profile as measured by AUC at 24 hours after an initial dose of the composition that is at least greater than about 50% of the total blood plasma concentration as measured by AUC at 24 hours of an initial dose of an orally administered solution comprising the at least one active pharmaceutical ingredient.

In a first embodiment of the sixth aspect the active pharmaceutical ingredient exhibits a significant food effect.

In a second embodiment of the sixth aspect the at least one active pharmaceutical ingredient is included in the range from about 0.1 to about 60% w/w.

In a seventh aspect the present disclosure provides a method of administering a formulation comprising orally administering to a fasted mammalian subject the formulation comprising Compound (I) having the formula

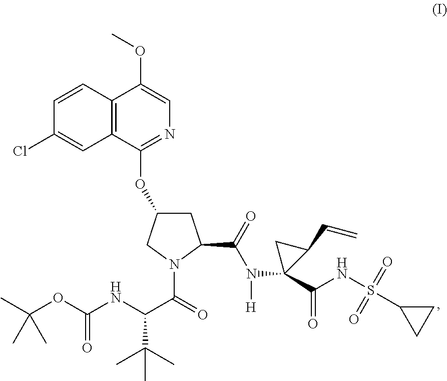 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

and has a fed to fasted ratio lower than at least about 2.

In a first embodiment of the seventh aspect the fed to fasted ratio is less than about 1.5.

In a second embodiment of the seventh aspect the fed to fasted ratio is less than about 1.0.

In a third embodiment of the seventh aspect the fed to fasted ratio is less than about 0.75.

In a fourth embodiment of the seventh aspect the formulation is in a form of a capsule.

In a fifth embodiment of the seventh aspect the formulation is in a form of a solubilized capsule.

In an eighth aspect the present disclosure provides a method of treating an HCV infection comprising the step of administering to a subject in need thereof a therapeutically effective amount of a formulation comprising a capsule comprising at least one active pharmaceutical ingredient comprising a solubilized compound having the formula (I)

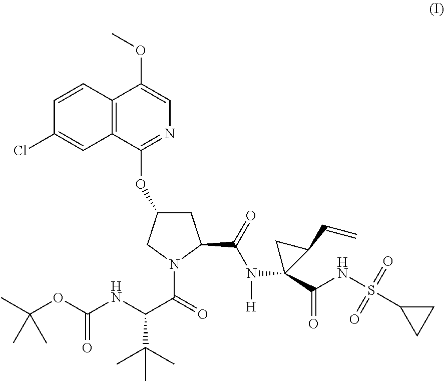 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

In a ninth aspect the present disclosure provides a method of treating an HCV infection comprising the step of administering to a subject in need thereof a therapeutically effective amount of a formulation comprising:

(a) at least one active pharmaceutical ingredient wherein the at least one active pharmaceutical ingredient comprises a compound of formula (I)

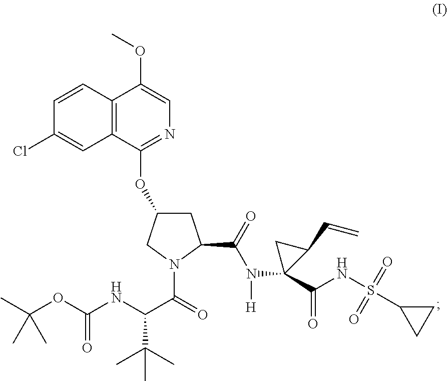 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

(b) at least one solubilizer selected from medium-chain fatty acid triglycerides, polyoxyethylated glycerides, a combination of medium-chain fatty acid mono- and diglycerides, and combinations thereof;

(c) at least one surfactant which is polyoxyethylene sorbitan monooleate; and

(d) at least one stabilizer which is butylated hydroxytoluene.

In a tenth aspect the present disclosure provides a method of treating an HCV infection comprising the step of administering to a subject in need thereof a formulation comprising a capsule comprising at least one active pharmaceutical ingredient comprising a solubilized compound having the formula (I)

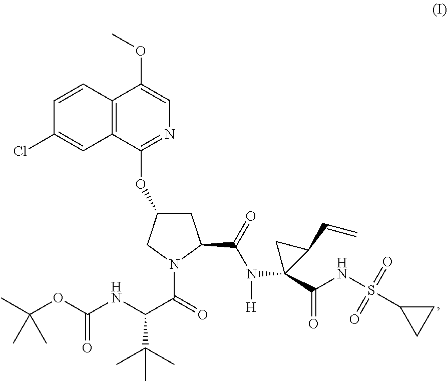 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

wherein the total dose of the compound of formula (I) administered to the subject is about 200 mg a day. In a first embodiment of the tenth aspect the compound of formula (I) is administered to the subject in doses of 100 mg two times a day. In a second embodiment of the tenth aspect the compound of formula (I) is administered to the subject in a dose of 100 mg one time a day.

In an eleventh aspect the present disclosure provides a method of treating an HCV infection comprising the step of administering to a subject in need thereof a formulation comprising a capsule comprising at least one active pharmaceutical ingredient comprising a solubilized compound having the formula (I)

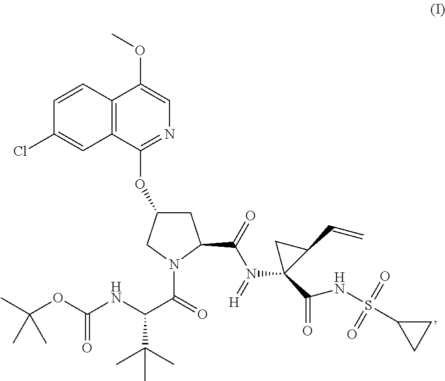

wherein the total dose of the compound of formula (I) administered to the subject is about 150 mg a day.

In a twelfth aspect the present disclosure provides a method of treating an HCV infection comprising the step of administering to a subject in need thereof a formulation comprising a capsule comprising at least one active pharmaceutical ingredient comprising a solubilized compound having the formula (I)

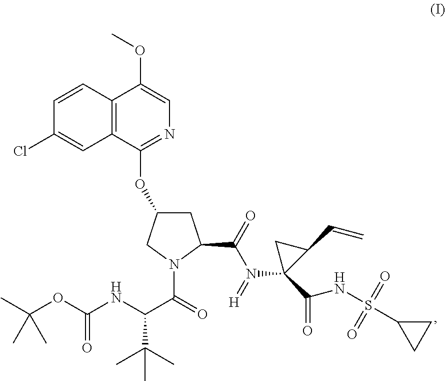

wherein the total dose of the compound of formula (I) administered to the subject is about 100 mg a day.

In a thirteenth aspect the present disclosure provides a method of treating an HCV infection comprising the step of administering to a subject in need thereof a formulation comprising a capsule comprising at least one active pharmaceutical ingredient comprising a solubilized compound having the formula (I)

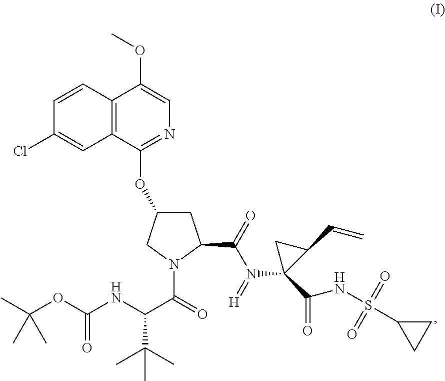

wherein the total dose of the compound of formula (I) administered to the subject is about 50 mg a day.

Other aspects of the present disclosure may include suitable combinations of embodiments disclosed herein.

Yet other aspects and embodiments may be found in the description provided herein.

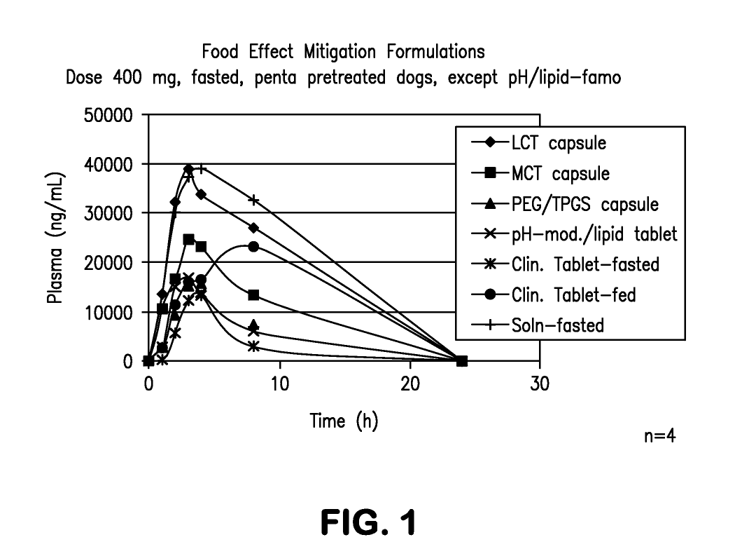

FIG. 1 illustrates the food effect in dogs of six different formulations of asunaprevir.

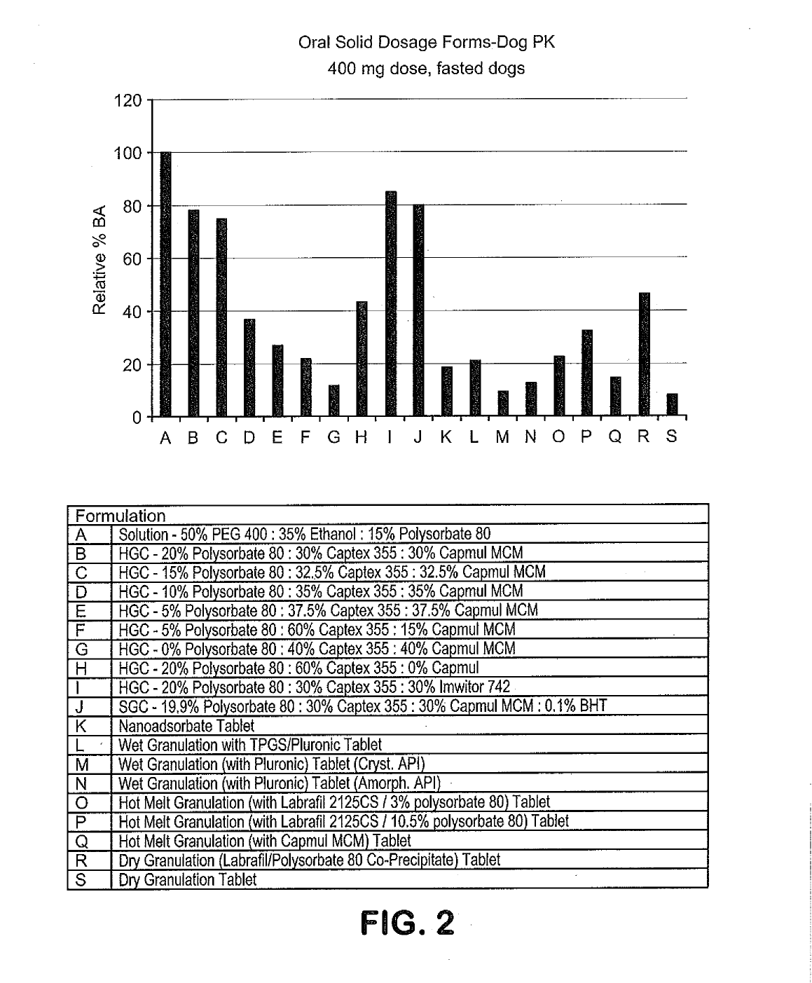

FIG. 2 illustrates the pharmacokinetics/bioavailability of capsule and tablet formulations of asunaprevir in dogs.

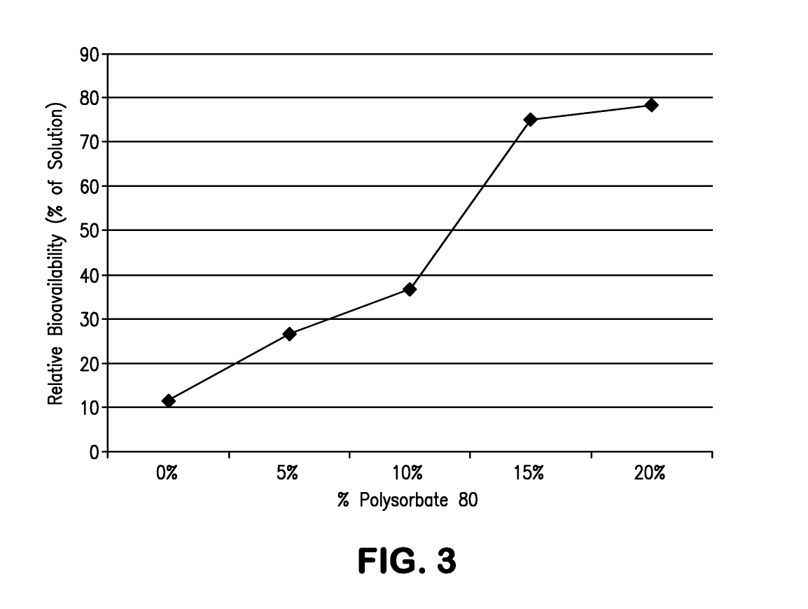

FIG. 3 illustrates the bioavailability in dogs of soft gel capsule formulations of asunaprevir with varying amounts of polysorbate 80.

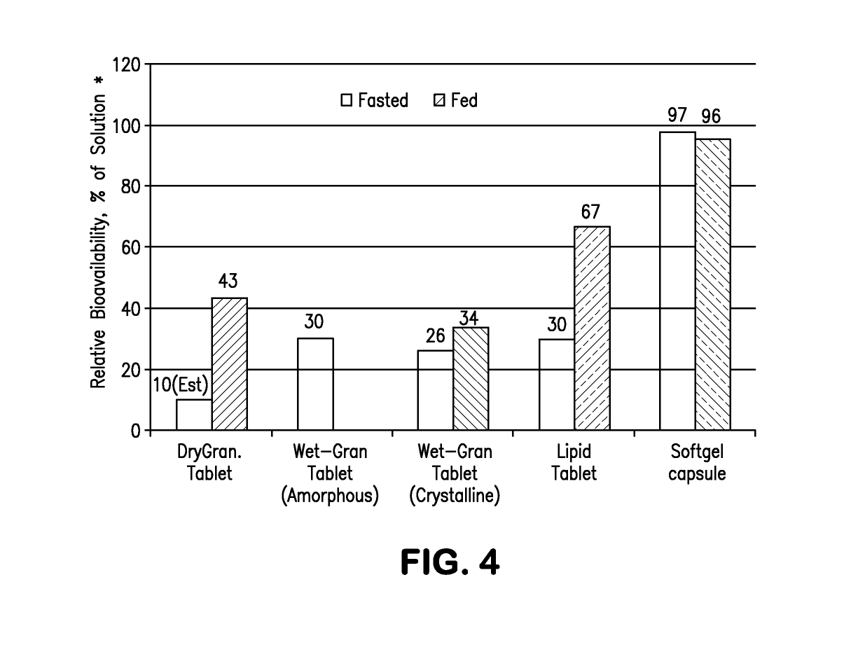

FIG. 4 illustrates the relative bioavailability of asunaprevir formulations in fasted and fed humans.

As used in the present specification, the following terms have the meanings indicated:

The term “therapeutically effective amount,” as used herein, refers to the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously.

The term “treating” refers to: (i) preventing a disease, disorder or condition from occurring in a patient that may be predisposed to the disease, disorder, and/or condition but has not yet been diagnosed as having it; (ii) inhibiting the disease, disorder, or condition, i.e., arresting its development; and (iii) relieving the disease, disorder, or condition, i.e., causing regression of the disease, disorder, and/or condition.

The terms “active pharmaceutical ingredient” and “API,” as used herein, refer to asunaprevir.

As used herein, the term “solubilizer” refers to any pharmaceutically acceptable agent that can dissolve API. In the present disclosure the solubilizer dissolves the API and forms the internal phase of an oil-in-water emulsion in which the API is incorporated in the oil droplet following aqueous dilution. Examples of solubilizers include medium-chain fatty acid triglycerides, combinations of mono, di and triglycerides of long-chain fatty acids; mono- and di-long-chain fatty acid esters of polyethylene glycol (commonly known as polyoxyethylated glycerides, i.e., oleoyl polyoxylglycerides and linoleoyl polyoxylglycerides); glycerol monocaprylocaprate, glycerol monocaprylate, glycerol mono/dicaprate, and the propylene glycol mono- and di-esters of medium-chain fatty acids such as propylene glycol monocaprylate, propylene glycol monolaurate, propylene glycol dilaurateand, and combinations thereof.

As used herein, the term “surfactant” refers to any pharmaceutically acceptable agent that provides a finer emulsion droplet size following aqueous dilution. Examples of surfactants include polyoxyethylene sorbitan monooleate; polyoxyethylated glycerides such as polyoxyl 35 castor oil and polyoxyl 40 hydrogenated castor oil; caprylocaproyl polyoxylglycerides (medium-chain fatty acid esters of polyethylene glycol 400 and medium-chain fatty acid esters of polyethylene glycol 300); and vitamin E TPGS (i.e., d-α-tocopheryl polyethylene glycol 1000 succinate.

As used herein, the term “stabilizer” refers to any pharmaceutically acceptable agent which minimizes oxidative pathways for the degradation of API. Examples of stabilizers include butylated hydroxytoluene (BHT); butylated hydroxyanisole (BHA); Vitamin E, propyl gallate; ascorbic acid; tert-butylhydroquinone (TBHQ); and the like.

The formulation solutions described herein may be encapsulated as a solution in soft or hard capsules manufactured from various materials including gelatin, hydroxypropyl methylcellulose (HPMC), cellulose, methylcellulose, starch, and other materials. The two-piece capsules may be banded, e.g., with a gelatin-based solution for hard gelatin capsules, or an HPMC-based solution for HPMC capsules. Soft gelatin capsule shells may contain one or more appropriate plasticizers such as glycerin, sorbitol, propylene glycol or others to impart suitable encapsulation, elasticity, mechanical strength, stability and dissolution properties. In addition, the hard or soft gelatin capsule shell may contain and/or be imprinted with various colorants and/or opacifiers.

The formulation solutions described herein may contain one or more of various flavoring agents (e.g., cherry, berry, mint, vanilla, and the like) and/or sweetening agents (e.g., sucrose, sorbitol, mannitol, fructose, dextrose, saccharin, aspartame, acesulfame potassium, and the like) to enhance palatability of the dosage form.

In another embodiment the present disclosure provides a composition comprising a therapeutically effective amount of a compound of formula (III)

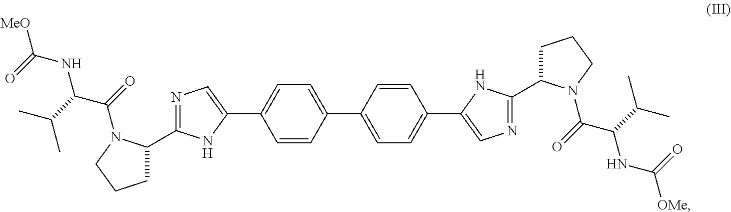

or a pharmaceutically acceptable salt thereof in combination with a formulation comprising a capsule comprising at least one active pharmaceutical ingredient comprising a solubilized compound of formula (I). In another embodiment the ratio of the compound of formula (III), or a pharmaceutically acceptable salt thereof, to the compound of formula (I) is between about 1:3 and about 3:1. In another embodiment the ratio of the compound of formula (III), or a pharmaceutically acceptable salt thereof, to the compound of formula (I) is between about 1:2.5 and about 2.5:1. In another embodiment the ratio of the compound of formula (III), or a pharmaceutically acceptable salt thereof, to the compound of formula (I) is about 1:1. In another embodiment the ratio of the compound of formula (III), or a pharmaceutically acceptable salt thereof, to the compound of formula (I) is about 1:2.5. In another embodiment the ratio of the compound of formula (III), or a pharmaceutically acceptable salt thereof, to the compound of formula (I) is about 2.5:1.

In another embodiment the composition comprising the compound of formula (III), or a pharmaceutically acceptable salt thereof, and the formulation of compound (I) further comprises one or two additional compounds having anti-HCV activity. Ion another embodiment at least one of the additional compounds is an interferon or a ribavirin. In another embodiment the interferon is selected from interferon alpha 2B, pegylated interferon alpha, consensus interferon, interferon alpha 2A, interferon lambda, pegylated interferon lambda, and lymphoblastoid interferon tau.

In another embodiment the present disclosure provides a composition comprising a therapeutically effective amount of a compound of formula (III), or a pharmaceutically acceptable salt thereof; a formulation comprising a capsule comprising at least one active pharmaceutical ingredient comprising a solubilized compound of formula (I); and a therapeutically effective amount of a compound effective to inhibit the function of HCV polymerase. In another embodiment the compound effective to inhibit the function of HCV polymerase is selected from PSI-6130, PF-00868554, ANA-598, IDX-375, IDX-184, INX-189, BI-207127, PSI-7851, PSI-938, PSI-879, VCH-759, VCH-916, VCH-222, BMS-929075, GS-9190, ABT-333, and ABT-072. In another embodiment the compound effective to inhibit the function of HCV nolvmerase is the comnound of formula (IV)

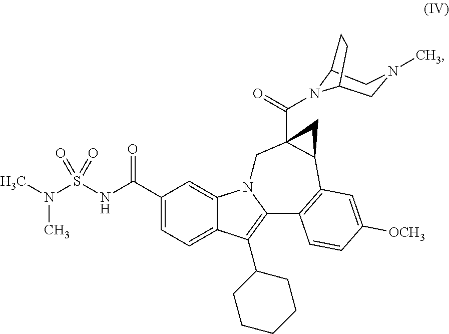 [SMILES: COc1ccc2c(c1)[C@@H]1C[C@]1(C(=O)N1C3CCC1CN(C)C3)Cn1c-2c(C2CCCCC2)c2ccc(C(=O)NS(=O)(=O)N(C)C)cc21]

or a pharmaceutically acceptable salt thereof.

In another embodiment the molar ratio of the compound of formula (III), or a pharmaceutically acceptable salt thereof, to the compound of formula (I), to the compound of formula (IV), or a pharmaceutically acceptable salt thereof, is about 1:20:5.

In another embodiment the molar ratio of the compound of formula (III), or a pharmaceutically acceptable salt thereof, to the compound of formula (I), to the compound of formula (IV), or a pharmaceutically acceptable salt thereof, is about 1:250:1000.

### EXAMPLES

The present disclosure will now be described in connection with certain embodiments which are not intended to limit its scope. On the contrary, the present disclosure covers all alternatives, modifications, and equivalents as can be included within the scope of the claims. Thus, the following examples, which include specific embodiments, will illustrate one practice of the present disclosure, it being understood that the examples are for the purposes of illustration of certain embodiments and are presented to provide what is believed to be the most useful and readily understood description of its procedures and conceptual aspects.

Asunaprevir can be prepared following the procedure described in U.S. Pat. No. 6,995,174, issued Feb. 7, 2006, which is incorporated herein in its entirety.

The following example is a typical procedure for manufacturing various aspects and embodiments of the present disclosure using mono-, di- and triglycerides of medium-chain fatty acids (e.g., CAPTEX® 355 and CAPMUL® MCM) with the surfactant polyoxyethylene sorbitan monooleate (polysorbate 80) and the antioxidant butylated hydroxytoluene:

1. Transfer the medium-chain triglyceride into the batching vessel.
2. Add the medium-chain mono/diglycerides and surfactant such as polyoxyethylene sorbitan monooleate (polysorbate 80) to the solution in Step 1.
3. Mix the batch from Step 2 to give a homogeneous solution.
4. Add butylated hydroxytoluene to the solution from Step 3 and mix to dissolve.
5. Add API to the solution in Step 4 and mix to dissolve.
6. Encapsulate the solution from Step 6 into a soft or hard gelatin capsules.
7. Dry the soft gelatin capsules to appropriate hardness or apply a band to the hard gelatin capsules.

### Example 1

Example 1

| Ingredient | mg/capsule | Percent (w/w) |
|---|---|---|
| API | 100 | 20 |
| Medium-Chain Triglycerides | 150 | 30 |
| Medium-Chain Mono/Diglycerides | 150 | 30 |
| Polyoxyethylene Sorbitan | 99.5 | 19.9 |
| Monooleate | | |
| Butylated Hydroxytoluene | 0.5 | 0.1 |
| Total | 500 | 100 |

The following example is a typical procedure for manufacturing various aspects and embodiments of the present disclosure using polyoxyethylated glycerides (linoleoyl polyoxylglycerides, e.g., LABRAFIL® 1944CS) with the surfactant polyoxyethylene sorbitan monooleate (Polysorbate 80) and the antioxidant butylated hydroxytoluene:

1. Transfer the oleoyl polyoxylglycerides into the batching vessel.
2. Add the surfactant such as polyoxyethylene sorbitan monooleate (polysorbate 80) to the solution in Step 1.
3. Mix the batch from Step 2 to give a homogeneous solution.
4. Add butylated hydroxytoluene to the solution from Step 3 and mix to dissolve.
5. Add API to the solution in Step 4 and mix to dissolve.
6. Encapsulate the solution from Step 6 into a soft or hard gelatin capsules.
7. Dry the soft gelatin capsules to appropriate hardness or apply a band to the hard gelatin capsules.

### Example 2

Example 2

| Ingredient | mg/capsule | Percent (w/w) |
|---|---|---|
| API | 100 | 20 |
| Oleoyl Polyoxylglycerides | 350 | 70 |
| Polyoxyethylene Sorbitan | 49.5 | 9.9 |
| Monooleate | | |
| Butylated Hydroxytoluene | 0.5 | 0.1 |
| Total | 500 | 100 |

An additional example of a formulation of the present disclosure using mono, di and triglycerides of medium-chain fatty acids (e.g., C® 355 and CAPMUL® MCM) with the surfactant Polyoxyl 35 Hydrogenated Castor Oil (e.g., Cremophor® EL) and the antioxidant butylated hydroxytoluene is shown in Example 3:

### Example 3

Example 3

| Ingredient | mg/capsule | Percent (w/w) |
|---|---|---|
| API | 100 | 20 |
| Medium-Chain Triglycerides | 150 | 30 |
| Medium-Chain Mono/Diglycerides | 150 | 30 |
| Polyoxyl 35 Hydrogenated Castor Oil | 99.5 | 19.9 |
| Butylated Hydroxytoluene | 0.5 | 0.1 |
| Total | 500 | 100 |

An additional example of a formulation of the present disclosure using triglycerides of medium-chain fatty acids (e.g., CAPTEX® 355) and a propylene glycol ester of a medium-chain fatty acid such as propylene glycol monocaprylate (e.g., CAPMUL® PG-8 or Capryol™ 90) with the surfactant polyoxyethylene sorbitan monooleate (Polysorbate 80) and the antioxidant butylated hydroxytoluene is shown in Example 4:

### Example 4

Example 4

| Ingredient | mg/capsule | Percent (w/w) |
|---|---|---|
| API | 100 | 20 |
| Medium-Chain Triglycerides | 200 | 40 |
| Propylene Glycol | 100 | 20 |
| Monocaprylate | | |
| Polyoxyethylene Sorbitan | 99.5 | 19.9 |
| Monooleate | | |
| Butylated Hydroxytoluene | 0.5 | 0.1 |
| Total | 500 | 100 |

An additional example of a formulation of the present disclosure using triglycerides of medium-chain fatty acids (e.g., CAPTEX® 355) and a propylene glycol ester of a medium-chain fatty acid such as propylene glycol monolaurate (e.g., CAPMUL® PG-12 or Lauroglycol™ 90) with the surfactant polyoxyethylene sorbitan monooleate (Polysorbate 80) and the antioxidant butylated hydroxytoluene is shown in Example 5:

### Example 5

Example 5

| Ingredient | mg/capsule | Percent (w/w) |
|---|---|---|
| API | 100 | 20 |
| Medium-Chain Triglycerides | 200 | 40 |
| Propylene Glycol | 100 | 20 |
| Monolaurate | | |
| Polyoxyethylene Sorbitan | 99.5 | 19.9 |
| Monooleate | | |
| Butylated Hydroxytoluene | 0.5 | 0.1 |
| Total | 500 | 100 |

An additional example of a formulation of the present disclosure using polyoxyethylated glycerides (linoleoyl polyoxylglycerides, e.g., LABRAFIL® 2125CS) with the surfactant polyoxyethylene sorbitan monooleate (Polysorbate 80) and the antioxidant butylated hydroxytoluene is shown in Example 6:

### Example 6

Example 6

| Ingredient | mg/capsule | Percent (w/w) |
|---|---|---|
| API | 100 | 20 |
| Linoleoyl Polyoxylglycerides | 350 | 70 |
| Polyoxyethylene Sorbitan | 49.5 | 9.9 |
| Monooleate | | |
| Butylated Hydroxytoluene | 0.5 | 0.1 |
| Total | 500 | 100 |

### Example 7

### In Vivo Studies

Table 1 and FIG. 1 present the results from a PK study conducted in dogs to compare the PK of four formulations developed in effort to increase the oral bioavailability of asunaprevir and mitigate the food effect observed for the dry granulation tablet formulation in humans. The capsule formulations represent three different solubilized approaches in which the drug was fully solubilized as a solution in either (1) a long-chain fatty acid triglyceride-based lipid self-emulsifying system containing 20% w/w drug load with LABRAFIL® M1944CS and polysorbate 80, designated as “LCT Capsule”, (2) a medium-chain triglyceride-based lipid self-emulsifying system containing 20% w/w drug load with CAPTEX® 355, CAPMUL® MCM and polysorbate 80, designated as “MCT Capsule”, and (3) a non-lipid system containing 20% w/w drug load with polyethylene glycol 3350 and vitamin E TPGS (i.e., d-α-tocopheryl polyethylene glycol 1000 succinate). Each capsule formulation was administered at a 400-mg dose to fasted dogs pre-treated with pentagastrin. In addition, a tablet formulation was evaluated containing 50% w/w drug load with Gelucire® 53/10 and the pH-modifier sodium bicarbonate. The tablet was administered at a 400-mg dose to fasted dogs pre-treated with famotidine to increase the pH of the GI tract. The PK of these 4 formulations was compared to the dry granulation tablet administered at a 400-mg dose to fasted dogs pre-treated with pentagastrin in one study arm, and at a 400-mg dose to fed dogs pre-treated with pentagastrin in a second study arm. Each of 4 dogs received all 6 formulation treatments (one week washout period before dosing each subsequent formulation).

As shown in Table 1 and FIG. 1, the long-chain fatty acid triglyceride-based lipid (LCT Capsule) and medium-chain triglyceride-based lipid (MCT Capsule) self-emulsifying systems provided superior oral absorption of asunaprevir in the dogs, increasing the oral bioavailability in the fasted-state by 3- to 5-fold. These LCT and MCT lipid-based solubilized capsule formulations also provided the most pronounced reduction in the significant food effect observed for the clinical dry granulation tablet formulation as represented by the ratio of the AUC for the formulation dosed in the fasted-state compared to the AUC for the tablet dosed in the fed-state.

TABLE 1
Dog PK Results from Formulation Screening Study

| | Pre- | Cmax (ng/mL) | | Tmax (h) | AUC-24 h (ng · h/mL) | | BA (%) | | Fed:Fasted |
|---|---|---|---|---|---|---|---|---|---|
| Formulation | Treatment | Mean | SD | Median | Mean | SD | Mean | SD | Ratio |
| HGC - 70% | Pentagastrin | 43126 | 13761 | 2.5 | 440671 | 391699 | 528 | 470 | 0.69 |
| LABRAFIL | & Fasted | | | | | | | | |
| M1944CS: 10% | | | | | | | | | |
| Polysorbate 80 | | | | | | | | | |
| (LCT Capsule) | | | | | | | | | |
| HGC - 20% | Pentagastrin | 29033 | 21362 | 2.5 | 243511 | 219862 | 292 | 264 | 1.25 |
| Polysorbate 80: | & Fasted | | | | | | | | |
| 30% CAPTEX 355: | | | | | | | | | |
| 30% CAPMUL MCM | | | | | | | | | |
| (MCT Capsule) | | | | | | | | | |
| HGC - 40% | Pentagastrin | 21180 | 6122 | 3 | 143844 | 77103 | 173 | 92 | 2.11 |
| PEG 3350: | & Fasted | | | | | | | | |
| 40% TPGS | | | | | | | | | |
| (PEG/TPGS | | | | | | | | | |
| Capsule) | | | | | | | | | |
| Dry Granulation | Famotidine | 19542 | 8756 | 2.5 | 131503 | 94509 | 158 | 113 | 2.31 |
| Tablet: Gelucire | & Fasted | | | | | | | | |
| 53/10: Sodium | | | | | | | | | |
| Bicarbonate | | | | | | | | | |
| (pH-Mod./Lipid | | | | | | | | | |
| Tablet) | | | | | | | | | |
| Dry Granulation | Pentagastrin | 14420 | 8305 | 4 | 83385 | 59101 | | | 3.64 |
| Tablet (Clin. | & Fasted | | | | | | | | |
| Tablet - Fasted) | | | | | | | | | |
| Dry Granulation | Pentagastrin | 29924 | 5570 | 6 | 303526 | 97245 | 364 | 117 | |
| Tablet (Clin. | & Fed | | | | | | | | |
| Tablet - Fed) | | | | | | | | | |

Table 2 and FIG. 2 present the cumulative results from several studies conducted in dogs to assess the PK of numerous capsule and tablet formulations of asunaprevir developed in an effort to increase the oral bioavailability and mitigate the food effect observed for the dry granulation tablet formulation in humans. All PK results shown were generated in pentagastrin-treated and fasted dogs dosed 400-mg of asunaprevir from the various formulations.

As noted, eight tablet formulations utilizing various approaches were developed and evaluated in an effort to increase the bioavailability of asunaprevir and mitigate the significant food effect observed for the dry granulation tablet. Compared to the dry granulation tablet which demonstrates a bioavailability of 8% relative to the solution formulation, some improvement in PK was observed for the various alternative tablet approaches, with relative bioavailabilities ranging from 9.1 to 46.5%.

Several variations of the medium-chain triglyceride-based lipid self-emulsifying system were evaluated to optimize bioavailability from the solubilized capsule approach. As shown in Table 2 and FIG. 2, a formulation composition containing 20% w/w drug load with 30% w/w medium-chain triglyceride (e.g., CAPTEX® 355), 30% w/w medium-chain mono/diglycerides (e.g., CAPMUL® MCM or Imwitor® 742) and 15-20% w/w polysorbate 80 provided the highest bioavailability of asunaprevir (relative to solution formulation) at 75-85%. As also noted in Table 2 and FIG. 3, this formulation encapsulated in either a hard gelatin capsule or soft gelatin capsule provided comparably superior PK results.

Unexpectedly, although there was no significant difference in drug solubility, ease of self-emulsification or emulsion droplet size distribution following aqueous dilution observed with the medium-chain triglyceride based system containing lower than 15% w/w polysorbate 80, the systems with 15% or 20% polysorbate 80 provided the highest relative bioavailabilities as shown in Table 2 and FIGS. 2 and 3.

TABLE 2
Cumulative Dog PK Results from Formulation Study

| | Pre- | Cmax (ng/mL) | | Tmax (h) | AUC-24 h (ng · h/mL) | | BA (%) | |
|---|---|---|---|---|---|---|---|---|
| Formulation | Treatment | Mean | SD | Median | Mean | SD | Mean | SD |
| Solution - 50% | Pentagstrin | 43126 | 13761 | 3 | 504019 | 265726 | 100 | 52.7 |
| PEG 400: 35% | & Fasted | | | | | | | |
| Ethanol: 15% | | | | | | | | |
| Polysorbate 80 | | | | | | | | |
| HGC - 20% | Pentagstrin | 40183 | 10619 | 4 | 395137 | 173805 | 78.4 | 34.5 |
| Polysorbate 80: | & Fasted | | | | | | | |
| 30% CAPTEX | | | | | | | | |
| 355: 30% | | | | | | | | |
| CAPMUL MCM | | | | | | | | |
| HGC - 15% | Pentagstrin | 40333 | 8396 | 3 | 378133 | 136931 | 75.0 | 27.2 |
| Polysorbate 80: | & Fasted | | | | | | | |
| 32.5% CAPTEX | | | | | | | | |
| 355: 32.5% | | | | | | | | |
| CAPMUL MCM | | | | | | | | |
| HGC - 10% | Pentagstrin | 24125 | 8424 | 3 | 185419 | 112587 | 36.8 | 22.3 |
| Polysorbate 80: | & Fasted | | | | | | | |
| 35% CAPTEX | | | | | | | | |
| 355: 35% | | | | | | | | |
| CAPMUL MCM | | | | | | | | |
| HGC - 5% | Pentagstrin | 20100 | 11392 | 3 | 134178 | 83467 | 26.6 | 16.6 |
| Polysorbate 80: | & Fasted | | | | | | | |
| 37.5% CAPTEX | | | | | | | | |
| 355: 37.5% | | | | | | | | |
| CAPMUL MCM | | | | | | | | |
| HGC - 5% | Pentagstrin | 15366 | 7695 | 2 | 109207 | 86876 | 21.7 | 17.2 |
| Polysorbate 80: | & Fasted | | | | | | | |
| 60% CAPTEX | | | | | | | | |
| 355: 15% | | | | | | | | |
| CAPMUL MCM) | | | | | | | | |
| HGC - 0% | Pentagstrin | 10066 | 4026 | 3 | 58241 | 25819 | 11.6 | 5.1 |
| Polysorbate 80: | & Fasted | | | | | | | |
| 40% CAPTEX | | | | | | | | |
| 355: 40% | | | | | | | | |
| CAPMUL MCM) | | | | | | | | |
| HGC - 20% | Pentagstrin | 26400 | 4359 | 4 | 217742 | 33535 | 43.2 | 6.7 |
| Polysorbate 80: | & Fasted | | | | | | | |
| 60% CAPTEX | | | | | | | | |
| 355: 0% CAPMUL | | | | | | | | |
| HGC - 20% | Pentagstrin | 41025 | 4613 | 3 | 429537 | 102361 | 85.2 | 20.3 |
| Polysorbate 80: | & Fasted | | | | | | | |
| 30% CAPTEX | | | | | | | | |
| 355: 30% | | | | | | | | |
| Imwitor 742 | | | | | | | | |
| SGC - 19.9% | Pentagstrin | 42675 | 15966 | 3 | 402129 | 183224 | 79.8 | 36.4 |
| Polysorbate 80: | & Fasted | | | | | | | |
| 30% CAPTEX: | | | | | | | | |
| 355 30% | | | | | | | | |
| CAPMUL MCM: | | | | | | | | |
| 0.1% BHT | | | | | | | | |
| Nanoadsorbate | Pentagstrin | 14367 | 4477 | 2 | 92814 | 51633 | 18.4 | 10.2 |
| Tablet - | & Fasted | | | | | | | |
| PVP: K12: TPGS: | | | | | | | | |
| Cabosil | | | | | | | | |
| Wet Granulation | Pentagstrin | 14382 | 3776 | 3 | 104507 | 28685 | 20.7 | 5.7 |
| with TPGS/ | & Fasted | | | | | | | |
| Pluronic Tablet | | | | | | | | |
| Wet Granulation | Pentagstrin | 8945 | 6744 | 3 | 45712 | 38370 | 9.1 | 7.6 |
| (with Pluronic) | & Fasted | | | | | | | |
| Tablet (Cryst. | | | | | | | | |
| API) | | | | | | | | |
| Wet Granulation | Pentagstrin | 10402 | 4941 | 2.5 | 63446 | 31814 | 12.6 | 6.3 |
| (with Pluronic) | & Fasted | | | | | | | |
| Tablet (Amorph. | | | | | | | | |
| API) | | | | | | | | |
| Hot Melt | Pentagstrin | 17376 | 3218 | 3 | 112885 | 34533 | 22.4 | 6.9 |
| Granulation (with | & Fasted | | | | | | | |
| LABRAFIL | | | | | | | | |
| 2125CS/3% | | | | | | | | |
| Polysorbate 80) | | | | | | | | |
| Tablet | | | | | | | | |
| Hot Melt | Pentagstrin | 20298 | 3551 | 2.5 | 162902 | 62210 | 32.3 | 12.3 |
| Granulation (with | & Fasted | | | | | | | |
| LABRAFIL | | | | | | | | |
| 2125CS/10.5% | | | | | | | | |
| Polysorbate 80) | | | | | | | | |
| Tablet | | | | | | | | |
| Hot Melt | Pentagstrin | 10655 | 2501 | 3 | 72608 | 20277 | 14.4 | 4.0 |
| Granulation (with | & Fasted | | | | | | | |
| CAPMUL MCM) | | | | | | | | |
| Tablet | | | | | | | | |
| Dry Granulation | Pentagstrin | 28844 | 8503 | 3 | 234373 | 87155 | 46.5 | 17.3 |
| (LABRAFIL/Poly- | & Fasted | | | | | | | |
| sorbate 80 | | | | | | | | |
| Co-Precipitate) | | | | | | | | |
| Tablet | | | | | | | | |
| Dry Granulation | Pentagstrin | 8694 | 586 | 2.5 | 40519 | 8796 | 8.0 | 1.8 |
| Tablet | & Fasted | | | | | | | |

FIG. 4 and Tables 3 and 4 present the results from a relative bioavailability clinical study conducted in humans to evaluate the PK and effect of fasted versus fed conditions on the oral absorption of various formulations of asunaprevir. The formulations evaluated included (1) film-coated 200-mg strength tablet manufactured from a dry granulation containing 50% w/w drug load with microcrystalline cellulose, croscarmellose sodium, sodium lauryl sulfate and magnesium stearate and film-coated with Opadry®-II (a white non-functional coating system), (2) film-coated 200-mg strength tablet manufactured from a wet granulation containing 50% w/w drug load (amorphous API) with microcrystalline cellulose, lactose monohydrate, croscarmellose sodium, polaxamer 188, vitamin E polyethylene glycol succinate and magnesium stearate) and film-coated with Opadry®-II, (3) film-coated 200-mg strength tablet manufactured from a wet granulation containing 50% w/w drug load (crystalline API) with microcrystalline cellulose, lactose monohydrate, croscarmellose sodium, polaxamer 188, vitamin E polyethylene glycol succinate and magnesium stearate) and film-coated with Opadry®-II, (4) uncoated 100-mg strength tablet manufactured from a dry granulation containing microcrystalline cellulose, croscarmellose sodium, linoleoyl polyoxyglycerides, polysorbate 80, magnesium aluminometasilicate, anhydrous dibasic calcium phosphate, magnesium stearate, and butylated hydroxytoluene (BHT), and (5) 100-mg strength soft gelatin capsule containing 20% w/w drug load dissolved in a mixture of medium-chain triglycerides, glycerol monocaprylocaprate, polysorbate 80, and butylated hydroxytoluene (BHT).

The study design included randomization of eight formulation/dietary-state (fed vs. fasted) treatments (A, B, C, D, E, F, G and H), with each of 35 subjects randomized to receive five single doses consisting of the 200-mg asunaprevir dry granulation tablet fed-state “A” (Reference) and four additional formulation/dietary-state treatments either fed (standard meal) or fasted, randomized from B-H. Single-dose pharmacokinetic (PK) parameters were derived from the plasma concentration versus time profiles. Relative bioavailabilities as shown in FIG. 4 and Tables 3 and 4 are calculated from the AUC(INF) values for each treatment compared to the AUC(INF) value of a solution formulation of asunaprevir (20 mg/mL asunaprevir in 50% w/w PEG 400/35% w/w Ethanol/15% w/w Polysorbate 80) dosed at 200-mg in a separate clinical study. The relative bioavailability of the reference dry granulation tablet dosed at 200-mg in the fasted-state was estimated from PK data also generated in a separate clinical study.

As shown in FIG. 3 and Table 3, the bioavailability of asunaprevir from the soft gelatin capsule in the fasted-state was approximately 3 to 10-fold higher compared to the tablet formulations. In addition, as shown in FIG. 3 and Table 4, the bioavailability from the soft gelatin capsule in the fed-state was approximately 1.4 to 2.8-fold higher compared to the tablet formulations. Furthermore, the results indicated substantially complete mitigation of food effect for the soft gelatin capsule formulation in contrast to the significant effect of food on oral bioavailability demonstrated by the tablet formulations. Substantially complete mitigation of the food effect is defined as a formulation that has a fed fasted ratio of 1.5 or less; or 1.25 or lower or 1.0 or lower.

TABLE 3
Results from Human Bioavailability Study

| | Treatment | | | | |
|---|---|---|---|---|---|
| | B: | | | | |
| | Wet | | | | |
| | Granulation | Treatment C: | | | |
| | Tablet | Wet Granulation | | Treatment G: | |
| PK | (Amorphous | Tablet | Treatment E: | Soft Gelatin | |
| Parameter | API) | (Crystalline API) | Lipid Tablet | Capsule | Solution* |
| Cmax - | 45.7 (160) | 40.9 (128) | 42 (87) | 252 (106) | 367 (72) |
| GM (CV %) | | | | | |
| AUCinf- | 328 (123) | 285 (88) | 324 (81) | 1060 (70) | 1087 (63) |
| GM (CV %) | | | | | |
| AUCinf | 30% | 26% | 30% | 97% | |
| Relative to | | | | | |
| Solution | | | | | |

Treatment B: 1 × 200 mg Asunaprevir Wet Granulation Tablet with Amorphous API (Fasted)
Treatment C: 1 × 200 mg Asunaprevir Wet Granulation Tablet with Crystalline API (Fasted)
Treatment E: 1 × 200 mg Asunaprevir Lipid Tablet (Fasted)
Treatment G: 1 × 200 mg Asunaprevir Soft Gelatin Capsule (Fasted)
*Solution: (Formulation: 20 mg/mL asunaprevir in 50% w/w PEG 400/35% w/w Ethanol/15% w/w Polysorbate 80

TABLE 4
Results from Human Bioavailability Study

| | Treatment A: | Treatment D: | | | |
|---|---|---|---|---|---|
| | Dry | Wet Granulation | | Treatment H: | |
| | Granulation | Tablet | Treatment F: | Soft Gelatin | |
| PK Parameter | Tablet | (Crystalline API) | Lipid Tablet | Capsule | Solution* |
| Cmax -- | 61.8 (97) | 51.9 (101) | 114 (77) | 288 (59) | 367 (72) |
| GM (CV %) | | | | | |
| AUCinf-- | 470 (61) | 366 (66) | 725 (53) | 1040 (41) | 1087 (63) |
| GM (CV %) | | | | | |
| AUCinf | 43% | 34% | 67% | 96% | |
| Relative to | | | | | |
| Solution | | | | | |

Treatment A: 1 × 200 mg Asunaprevir Dry Granulation Tablet (Fed)
Treatment D: 1 × 200 mg Asunaprevir Wet Granulation Tablet with Crystalline API (Fed)
Treatment F: 1 × 200 mg Asunaprevir Lipid Tablet (Fed)
Treatment H: 1 × 200 mg Asunaprevir Soft Gelatin Capsule (Fed)
*Solution: PK data from separate clinical study (Formulation: 20 mg/mL asunaprevir in 50% w/w PEG 400/35% w/w Ethanol/15% w/w Polysorbate 80

Table 5 shows the chemical stability of asunaprevir in the formulated capsule with varying amounts of BHT. As shown below, using as little as 0.02% BHT provides suitable chemical stability with less than 2% degradation, including at least 6-months at accelerated stability conditions such as 25° C./60% RH (open dish) and 40° C./75% RH in various HDPE bottles and blister packages.

TABLE 5
Effect of Butylated Hydroxytoluene (BHT) Level
on the Stability of a Formulation Solution

| | | 6 Months 50° C. | | | | | |
|---|---|---|---|---|---|---|---|
| | | (Solution-in-Vial) | | 6 Months 40° C./75% RH (Capsule) | | | |
| | | API | | API | | | |
| | | Potency | % Deg. | Potency | % Deg. | % Deg. | % Deg. |
| Formulation | % BHT | Retention | #1 | Retention | #1 | #2 | #3 |
| 20% API | 0.1 | 103.1%* | <0.05* | 99.5%* | <0.05* | 0.15* | 0.18* |
| 30% Medium-Chain | | | | | | | |
| Triglycerides | | | | | | | |
| 30% Medium-Chain | | | | | | | |
| Mono/Diglycerides | | | | | | | |
| 19.9% Polysorbate 80 | | | | | | | |
| 20% API | 0.05 | 100.0% | <0.05 | 99.1% | <0.05 | 0.15 | 0.18 |
| 30% Medium-Chain | | | | | | | |
| Triglycerides | | | | | | | |
| 30% Medium-Chain | | | | | | | |
| Mono/Diglycerides | | | | | | | |
| 19.95% Polysorbate 80 | | | | | | | |
| 20% API | 0.02 | 98.7% | <0.05 | 98.4% | <0.05 | 0.15 | 0.19 |
| 30% Medium-Chain | | | | | | | |
| Triglycerides | | | | | | | |
| 30% Medium-Chain | | | | | | | |
| Mono/Diglycerides | | | | | | | |
| 19.98% Polysorbate 80 | | | | | | | |
| 20% API | 0.01 | 73.9% | 14.2 | 98.7% | <0.05 | 0.14 | 0.19 |
| 30% Medium-Chain | | | | | | | |
| Triglycerides | | | | | | | |
| 30% Medium-Chain | | | | | | | |
| Mono/Diglycerides | | | | | | | |
| 19.99% Polysorbate 80 | | | | | | | |
| 20% API | 0 | 3.8% | 68.4 | 86.0% | 15.1 | 0.75 | 0.38 |
| 30% Medium-Chain | | | | | | | |
| Triglycerides | | | | | | | |
| 30% Medium-Chain | | | | | | | |
| Mono/Diglycerides | | | | | | | |
| 20% Polysorbate 80 | | | | | | | |

*Average of 7 formulations tested

### Dosing Schedule Studies

A randomized 8-treatment, 5-period incomplete crossover study balanced for first-order carryover effects, was conducted in healthy men and women ages 18-49 years and BMI 18-32 kg/m². Subjects (n=35) each received 200 mg doses of the reference dry-granulated tablet formulation with standard meal and four of the following test treatments: formulations 1, 2, 3, and softgel capsule administered fasted and formulations 2, 3, and softgel capsule administered with standard meal. Subjects had periodic safety assessments and were monitored for adverse events (AEs). Blood samples for pharmacokinetics (PK) were collected for 72 h post-dose. Noncompartmental PK were derived. Geometric mean ratios (GMR) and 90% confidence intervals (CI) were calculated using general linear mixed-effect models. Post-hoc GMRs and 90% CIs were estimated for 100 mg of softgel capsule using normalized exposures from 200 mg data. Distributions of softgel capsule steady state AUC (AUCss) at 100 mg BID were simulated and evaluated in context of prior exposure-response and exposure-safety assessments.

All AEs were mild/moderate and no discontinuations were due to AEs. Formulations 1, 2, and 3 had lower bioavailability than the reference, unacceptable food effects or both. GMR [90% CI] for fasted softgel capsule was 2.23 [1.86-2.67] for AUC(0-∞) and 4.09[3.08-5.43] for Cmax. For softgel capsule with food, GMR [90% CI] was 2.60[2.17-3.19] for AUC(0-∞), indicating minimal food effect (only +17%), and 5.36[4.03-7.12] for Cmax. Post-hoc comparisons for normalized exposures of softgel capsule at 100 mg fasted relative to the 200 mg reference tablet, provided GMR [90% CI] of 1.11[0.929-1.34] for AUC(0-∞) and 2.04[1.54-2.72] for Cmax. For 100 mg softgel capsule fed, GMR [90% CI] was 1.30[1.09-1.56] for AUC(0-∞) and 2.68[2.02-3.56] for Cmax. Increased ASV Cmax values with softgel capsule were transient when fed or fasted. Simulations suggested considerable overlap of AUCss between projected softgel capsule exposures at 100 mg BID and fed tablet at 200 mg BID. Exposure-safety analysis indicated that the probability of ASV-associated ALT elevations would be expected to be similar with 100 mg BID softgel relative to 200 mg of tablet formulation. Higher but transient ASV Cmax values are unlikely to be clinically relevant as both efficacy and safety events for ASV appear to correlate best with AUCss.

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

What is claimed is:

1. A formulation comprising a capsule comprising at least one active pharmaceutical ingredient comprising a solubilized compound having the formula (I)

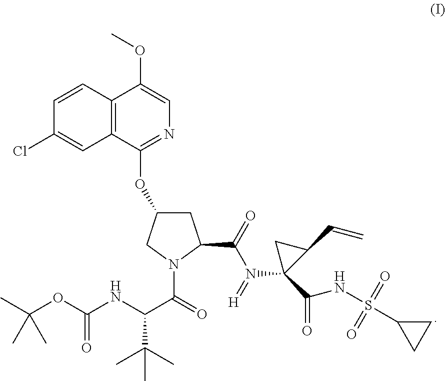

2. The formulation of claim 1, further comprising at least one solubilizer and optionally comprising at least one surfactant, and/or at least one stabilizer.

3. The formulation of claim 2, wherein the at least one stabilizer is included in the range from about 0.01 to about 1% w/w.

4. The formulation of claim 2 wherein the at least one active pharmaceutical ingredient is included in the range from about 0.1 to about 60% w/w, the at least one solubilizer is included in the range from about 1 to about 80% w/w, the at least one surfactant is included in the range from about 0 to about 60% w/w, and the at least one stabilizer is included in the range from about 0.01 to about 1% w/w.

5. The formulation of claim 2 wherein the at least one active pharmaceutical ingredient is included in the range from about 1 to about 40% w/w, the at least one solubilizer is included in the range from about 10 to about 80% w/w, the at least one surfactant is included in the range from about 5 to about 40% w/w, and the at least one stabilizer is included in the range from about 0.02 to about 0.5% w/w.

6. The formulation of claim 2 wherein the at least one active pharmaceutical ingredient is included in the range from about 1 to about 40% w/w, the at least one solubilizer is included in the range from about 5 to about 80% w/w, the at least one surfactant is included in the range from about 15 to about 40% w/w, and the at least one stabilizer is included in the range from about 0.05 to about 0.2% w/w.

7. The formulation of claim 2 wherein the at least one solubilizer is comprised of medium-chain fatty acid triglycerides and a combination of medium-chain fatty acid mono- and diglycerides.

8. The formulation of claim 2 wherein the at least one solubilizer is polyoxyethylated glycerides.

9. The formulation of claim 2 wherein the at least one surfactant is polyoxyethylene sorbitan monooleate.

10. The formulation of claim 2 wherein the at least one stabilizer is butylated hydroxytoluene.

11. A formulation comprising:
(a) at least one active pharmaceutical ingredient wherein the at least one active pharmaceutical ingredient comprises a compound of formula (I) 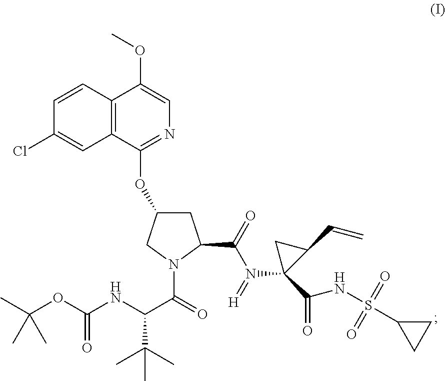
(b) at least one solubilizer selected from medium-chain fatty acid triglycerides, polyoxyethylated glycerides, a combination of medium-chain fatty acid mono- and diglycerides, and combinations thereof;
(c) at least one surfactant which is polyoxyethylene sorbitan monooleate; and
(d) at least one stabilizer which is butylated hydroxytoluene.

12. The formulation of claim 11 wherein the at least one active pharmaceutical ingredient is included in the range from about 1 to about 40% w/w.

13. The formulation of claim 11 wherein the at least one stabilizer is included in the range from about 0.05 to about 0.2% w/w.

14. The formulation of claim 11 wherein the at least one solubilizer is medium-chain fatty acid triglycerides and a combination of medium-chain fatty acid mono- and diglycerides.

15. The formulation of claim 14 wherein the at least one solubilizer is included in the range from about 10 to about 80% w/w.

16. The formulation of claim 15 wherein the at least one surfactant is included in the range from about 15 to about 40% w/w.

17. The formulation of claim 11 wherein the at least one solubilizer is polyoxyethylated glycerides.

18. The formulation of claim 17 wherein the at least one solubilizer is included in the range from about 1 to about 80% w/w.

19. The formulation of claim 17 wherein the at least one surfactant is included in the range from about 1 to about 40% w/w.

20. A formulation comprising a capsule comprising at least one active pharmaceutical ingredient comprising a solubilized compound having the formula (I) 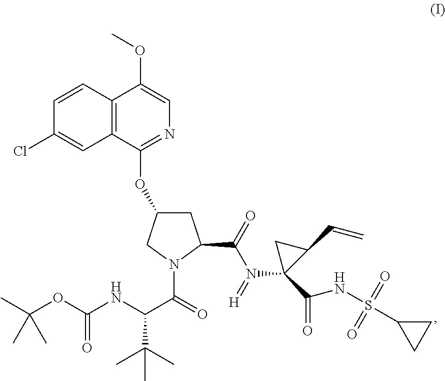
that is stable for at least six months at 40° C. and 75% relative humidity.

21. The formulation of claim 20, wherein the at least one active pharmaceutical ingredient has at least about 98% potency retention.

22. The formulation of claim 20, wherein the formulation has less than 10% total degradants.

23. The formulation of claim 20, wherein the formulation has less than 5% total degradants.

24. The formulation of claim 20, wherein the formulation has less than 2% total degradants.

25. The formulation of claim 20, wherein the formulation has less than 1% total degradants.

26. The formulation of claim 20, wherein the formulation has less than 0.5% total degradants.

27. The formulation of claim 20, wherein the formulation comprises at least one stabilizer in an amount from about 0.01 to about 1.0% w/w.

28. The formulation of claim 27, wherein the at least one stabilizer is selected from butylated hydroxytoluene, butylated hydroxyanisole, Vitamin E, propyl gallate, ascorbic acid, and tert-butylhydroquinone.

29. The formulation of claim 28, wherein the at least one stabilizer comprises butylated hydroxytoluene.

30. A method of administering a formulation comprising orally administering to a fasted or fed mammalian subject a formulation comprising Compound (I) having the formula: 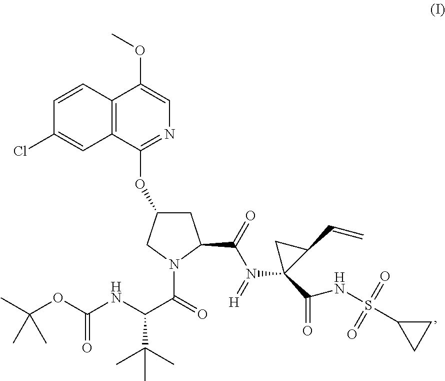
to provide a total blood plasma concentration profile of Compound (I), as measured by AUC at 24 hours after an initial dose of the composition, that is at least greater than about 50% of the total blood plasma concentration as measured by AUC at 24 hours of an initial dose of an orally administered solution comprising Compound (I).

31. The method of claim 30, wherein the AUC is at least greater than about 60% of the AUC at 24 hours of the solution when orally administered regardless if the subject is fasted or fed.

32. The method of claim 30, wherein the AUC is at least greater than about 70% of the AUC at 24 hours of the solution when orally administered regardless if the subject is fasted or fed.

33. The method of claim 30, wherein the AUC is at least greater than about 80% of the AUC at 24 hours of the solution when orally administered regardless if the subject is fasted or fed.

34. The method of claim 30, wherein the AUC is at least greater than about 90% of the AUC at 24 hours of the solution when orally administered regardless if the subject is fasted or fed.

35. The method of claim 30, wherein the formulation is in a form of a capsule.

36. The method of claim 30, wherein the formulation is in a form of a solubilized capsule.

37. A method of administering a formulation comprising orally administering to a fasted mammalian subject the composition comprising Compound (I) having the formula: 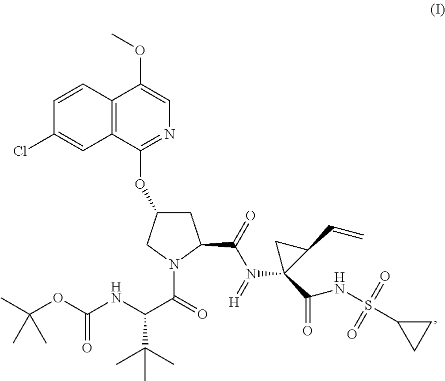
to provide a blood plasma concentration profile after an initial dose of the composition with a Cmax of Compound (I) after an initial dose of the composition that is at least greater than about 40% of the Cmax of an orally administered solution comprising Compound (I).

38. The method of claim 37, wherein the Cmax of the composition is at least or greater than about 50% of the Cmax of an orally administered solution.

39. The method of claim 37, wherein the Cmax of the composition is at least or greater than about 60% of the Cmax of an orally administered solution.

40. The method of claim 37, wherein the Tmax is at least about 3 hours.

41. The method of claim 37, wherein the formulation is in a form of a capsule.

42. The method of claim 37, wherein the formulation is in a form of a solubilized capsule.

43. A method of administering an oral solid dosage composition comprising orally administering to a fasted mammalian subject the composition comprising at least one poorly soluble active pharmaceutical ingredient to provide a total blood plasma concentration profile as measured by AUC at 24 hours after an initial dose of the composition that is at least greater than about 50% of the total blood plasma concentration as measured by AUC at 24 hours of an initial dose of an orally administered solution comprising the at least one active pharmaceutical ingredient.

44. The method of claim 43, wherein the active pharmaceutical ingredient exhibits a significant food effect.

45. The method of claim 43, wherein the at least one active pharmaceutical ingredient is included in the range from about 0.1 to about 60% w/w.

46. A method of administering a formulation comprising orally administering to a fasted mammalian subject the formulation comprising Compound (I) having the formula 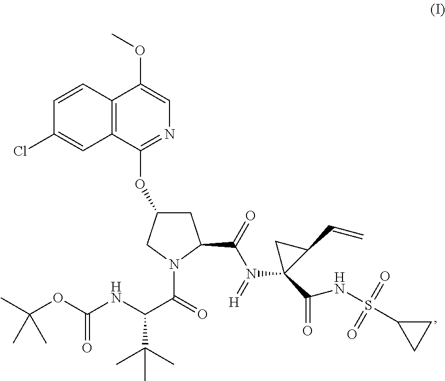
and has a fed to fasted ratio lower than at least about 2.

47. The method of claim 46, wherein the fed to fasted ratio is less than about 1.5.

48. The method of claim 46, wherein the fed to fasted ratio is less than about 1.0.

49. The method of claim 46, wherein the fed to fasted ratio is less than about 0.75.

50. The method of claim 46, wherein the formulation is in a form of a capsule.

51. The method of claim 46, wherein the formulation is in a form of a solubilized capsule.

52. A method of treating an HCV infection comprising the step of administering to a subject in need thereof a therapeutically effective amount of the formulation of claim 1.

53. A method of treating an HCV infection comprising the step of administering to a subject in need thereof a therapeutically effective amount of the formulation of claim 11.

54. A method of treating an HCV infection comprising the step of administering to a subject in need thereof a formulation comprising a capsule comprising at least one active pharmaceutical ingredient comprising a solubilized compound having the formula (I) 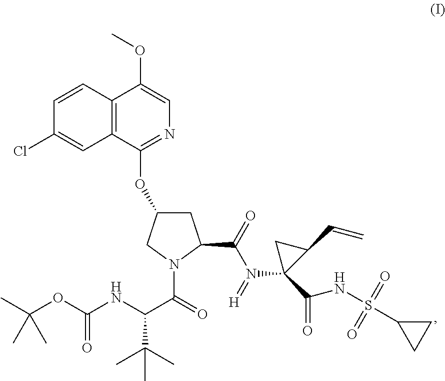
wherein the total dose of the compound of formula (I) administered to the subject is about 200 mg a day.

55. The method of claim 54 wherein the compound of formula (I) is administered to the subject in doses of 100 mg two times a day.

56. A method of treating an HCV infection comprising the step of administering to a subject in need thereof a formulation comprising a capsule comprising at least one active pharmaceutical ingredient comprising a solubilized compound having the formula (I) 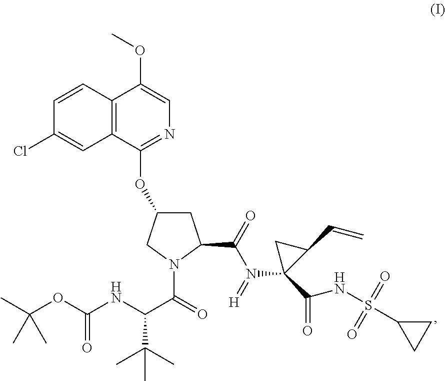
wherein the total dose of the compound of formula (I) administered to the subject is about 150 mg a day.

57. A method of treating an HCV infection comprising the step of administering to a subject in need thereof a formulation comprising a capsule comprising at least one active pharmaceutical ingredient comprising a solubilized compound having the formula (I) 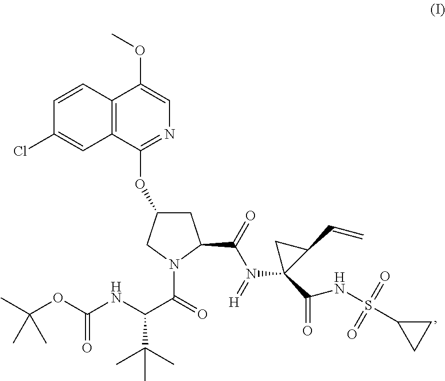
wherein the total dose of the compound of formula (I) administered to the subject is about 100 mg a day.

58. A method of treating an HCV infection comprising the step of administering to a subject in need thereof a formulation comprising a capsule comprising at least one active pharmaceutical ingredient comprising a solubilized compound having the formula (I) 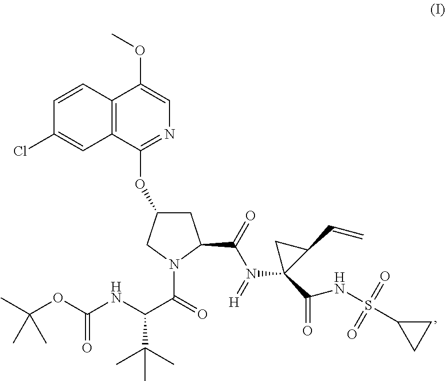
wherein the total dose of the compound of formula (I) administered to the subject is about 50 mg a day.
